(19) United States
(12) Patent Application Publication
(10) Pub. No.: US 2006/0127311 A1
(43) Pub. Date: Jun. 15, 2006

(54) Radiation dosimetry and blocking antibodies and methods and uses therefor in the treatment of cancer

(75) Inventors: David A. Rizzieri, Chapel Hill, NC (US); Darell D. Bigner, Mebane, NC (US); Michael R. Zalutsky, Chapel Hill, NC (US); Gamal Akabani-Hneide, Mexico City (MX)

Correspondence Address: DARBY & DARBY P.C., P. O. BOX 5257, NEW YORK, NY, 10150-5257, US

(73) Assignee: Duke University, Durham, NC (US)

(21) Appl. No.: 11/282,115

(22) Filed: Nov. 16, 2005

Related U.S. Application Data
(60) Provisional application No. 60/628,902, filed on Nov. 17, 2004.
(60) Provisional application No. 60/628,740, filed on Nov. 17, 2004.

(51) Int. Cl. A61K 51/00

(52) U.S. Cl. 424/1.49; 424/9.36

(57) ABSTRACT

Disclosed is a method for dosimetry estimation for a region of interest at or around a surgically created resection cavity in a subject. These methods enable medical practitioners to estimate the amount of administered Radioimmunotherapy (RIT) agent needed to safely and effectively achieve a final Radiation Absorbed Dose (RAD). Furthermore, computer hardware and software are provided herein, so that the methods according to the invention may be automated for more efficient use. Also disclosed is a method of enhancing delivery of therapeutic antibodies that specifically bind to an extracellular stromal constituent of a tumor in a mammalian subject. The method comprises administering to a subject an effective dosage of a blocking antibody, said blocking antibodies specifically binding to said extracellular stromal constituent and blocking the binding of therapeutic antibodies to non-target tissue.

This application claims the benefit of U.S. Provisional Patent Application No. 60/628,902, filed Nov. 17, 2004, and U.S. Provisional Patent Application No. 60/628,740, filed Nov. 17, 2004, which are hereby incorporated by reference in there entirety.

### GOVERNMENT SUPPORT

This invention was made with Government support under grant numbers MO1-RR 30, NS20023, CAI 1898, CA70164, CA42324, IP50CA108786-01, 5P20CA96890 and PDT-414 from the National Center for Research Resources General Clinical Research Centers Program, National Institutes of Health and the American Cancer Society. The Government has certain rights to this invention

### FIELD OF THE INVENTION

The present invention relates to increasing the safety and efficacy of methods of treating cancer with targeted therapy, e.g., radio-labeled therapeutic antibodies. For example, the invention provides for the use of dosimetry techniques to estimate dosages of radiation that are safe and therapeutically effective when administered to subjects in need of antibody therapy for the treatment of cancers and tumors. The present invention also relates to the use of unlabeled antibodies to block the binding of labeled therapeutic antibodies to healthy non-target tissue.

### BACKGROUND OF THE INVENTION

Therapy for subjects with tumors, e.g., brain tumors, generally includes a surgical resection of the tumor combined with external beam therapy and/or systemic chemotherapy. After surgical resection, a “surgically created resection cavity” (“SCRC”) remains where the tumor was removed. Radio-immunotherapy (“RIT”) may be administered to the SCRC, e.g. ,by using a reservoir and catheter arrangement, such as a Rickman reservoir, that is implanted near the SCRC. Typically, the reservoir is loaded with an RIT agent, e.g., a radio-labeled antibody. The RIT agent passes through the catheter and into the surgically created resection cavity. RIT agents are known in the art to be extremely toxic. Therefore, determining an accurate RIT dose that increases local tumor control while minimizing or reducing damage to normal brain tissue is important. It is critical, therefore, to accurately determine what RIT dose should be administered to a subject in need thereof, in order to achieve a final absorbed dose that is both effective and safe.

In subjects with Glioblastoma Multiforme (GBM) brain tumors, the median survival after diagnosis is between about 40 to 50 weeks. After surgical resection of the tumor, which leaves a cavity in the brain, between 90 to 95% of the progression and recurrence occurs locally within the margins of the resection cavity.

The treatment of human cancers with therapeutic antibodies is an emerging approach to this difficult disease. In the Unites States, two anti-CD20 radio-labeled murine monoclonal antibodies for the treatment of lymphoma have been approved: Zevalin™, produced by IDEC Pharmaceuticals, and Bexxar™, produced by Corixa Corporation. There nevertheless remains a need for additional methods for treating cancer, and particularly methods that would aid in increasing the specificity of treatment and decreasing undesired side effects of such treatments.

Bigner et al., U.S. Pat. No. 5,624,659, describes methods of treating solid and cystic tumors with the anti-tenascin monoclonal antibody 81C6. See also D. Bigner et al., J. Clin. Oncol. 16:2202-2212 (1998). See also I. Cokgor et al., J. Clin. Oncol. 18(22):3862-3872 (2000); D. Reardon et al., J. Clin. Oncol. 20(5):1389-1397 (2002). Rizzieri et al., U.S. Patent application Ser. No. 10/008,062, describes anti-tenascin monoclonal antibody therapy for the treatment of lymphoma. See also D. Rizzieri et al., Blood 104, 642-648 (2004); G. Akabani et al., Int. J. Radiat. Oncol. Biol. Phys. 46:947-958 (2000). Abrams et al., U.S. Pat. No. RE38,008, concerns methods of improved cell targeting of antibody, antibody fragments, hormones and other targeting agents, and conjugates thereof. In contrast, the present invention is concerned with antibodies targeted to extracellular stromal constituents and not to cells per se. The targeting of antibodies to stromal constituents is neither suggested nor described in Abrams.

Following administration of an RIT agent to a subject in need thereof, only a portion of the radiation energy is deposited, i.e., actually absorbed into animal or human tissue. This is known as the Radiation Absorbed Dose (“RAD”). The aforementioned references relate to: 1) the determination of dose-response relationships in patients with malignant brain tumors treated with ¹³¹I-labeled 81C6 monoclonal antibody; 2) the amount of radiation energy absorbed upon administration of radiation dosages that ranged from 20 to 180 mCi per patient with malignant brain tumors; 3) determination of the maximum tolerated dose (MTD) of ¹³¹I-labeled 81C6 monoclonal antibody administered into surgically created resection cavities of patients with malignant gliomas; 4) an assessment of the efficacy and toxicity of intra-resection cavity administration of 120 mCi of ¹³¹I-labeled 81 C6 monoclonal antibody in patients with malignant gliomas; and 5) a study of the pharmacokinetics, dosimetry, toxicity and response to human/mouse chimeric ¹³¹I-labeled 81C6 monoclonal antibody (ch81C6) treatment of lymphoma. However, they do not teach the medical practitioner how much RIT agent needs to be administered as a therapy dose to the subject in order to achieve a predetermined safe and effective RAD.

Another problem inherent in using RIT agents in the treatment of cancer is that a portion of the radiation energy from the administered RIT dose is actually deposited into healthy tissue rather than the diseased or cancerous area of interest. For example, monoclonal antibody 81 C6 is specific for the protein tenascin, which is expressed by both healthy and tumorous cells. Therefore, when ¹³¹I-labeled 81C6 is administered as an RIT agent, the 81C6 antibody facilitates delivery of the radionuclide (¹³¹I) to areas where tenascin is located—the antibody cannot distinguish between healthy and cancerous tissue. Therefore, some of the radiation is absorbed by healthy tissue, with two consequences: 1) radiation toxicity to healthy tissue; and 2) divergence of the RIT agent away from its intended target, which results in less effective therapy for the subject. Thus, there remains a need in the art for methods that enhance the delivery of an RIT agent to its intended target and avoid delivery of the RIT agent to healthy tissue. The blocking antibody methods according to the instant invention solve this problem.

### SUMMARY OF THE INVENTION

The present invention is directed to solving the aforementioned problems.

Specifically, the present invention enables medical practitioners to estimate the amount of administered RIT agent needed to achieve the final RAD. By delivering radio-labeled antibodies to SCRCs in escalating dosages and performing dose-response analyses according to the methods of the present invention, medical practitioners will be able to estimate the dosage amount of an RIT agent needed to achieve a pre-determined RAD. Furthermore, computer hardware and software are provided herein, to enable the methods according to the invention for more efficient use. In addition, the present invention includes blocking antibody methods that enhance the delivery of an RIT agent to its intended target and avoid delivery of the RIT agent to healthy tissue.

A first aspect of the invention relates to a method for dosimetry estimation for a region of interest (ROI) at or around a surgically created resection cavity (SCRC) in a subject, the method involving first determining a size of the SCRC, then administering a dosimetric dose of a radio-labeled antibody into the region of interest, then measuring detected radiation from the region of interest at various times subsequent to administering the dosimetric dose, then determining a residence time (i.e., how long the radio-labeled antibody remains in the body after administration) based on the measured size of the SCRC plus the measured detected radiation from the region of interest; and, finally, calculating the amount of radio-labeled antibody needed to be administered (the “radio-immunotherapeutic,” or “RIT,”) in order to achieve a predetermined radiation absorbed dose (“RAD”), which is the dose of radiation that needs to be absorbed by the diseased or cancerous tissue in order for the therapy to be effective.

Another aspect of this invention relates to using whole-body scintigraphy to detect radiation from the region of interest. A further aspect of this invention relates to instances when the whole-body scintigraphy is performed: first at the same time as administering the dosimetric dose, then at a second time that is about twenty-four hours after administration of the dosimetric dose, followed by a third time about forty-eight hours after administration of the dosimetric dose.

A further aspect of this invention comprises performing magnetic resonance imaging (“MRI”) to determine the size of the SCRC.

Another aspect of this invention relates to using experimental data to determine the optimal RAD needed for effective cancer therapy. A related aspect of this invention relates to when the predetermined absorbed dose is about 44 Gy.

Further aspects of this invention relate to the method wherein the administered therapeutic RIT dose is an amount of ¹³¹I-labeled anti-tenascin murine 81C6 (m81C6) monoclonal antibody or 131 I-labeled anti-tenascin human/murine chimeric 81 C6 (ch81C6) monoclonal antibody.

Yet another aspect of this invention relates to the method wherein the region of interest is a region of parenchyma about two centimeters from the margin of the resection cavity. A further aspect of this invention relates to the method wherein the administered RIT dose is calculated based on the following formula:

$${A}_{0} = \frac{{D}_{SCRC}}{S ( {B}_{2 - cm} ← SCRC ) {τ}_{SCRC}}$$

where D_{SCRC} is the predetermined absorbed dose, S(B₂₋ₘ→SCRC) is an estimated S-value based on the size of the resection cavity in Gy hr mCi-1, and τ_{SCRC} is the resection cavity residence time.

Another aspect of this invention relates to the method wherein the RIT dose is administered using a Rickman reservoir implantation.

Another aspect of this invention relates to the use of a radio-labeled antibody for the preparation of a medicament for use in a method of treatment of a region of interest at or around a surgically created resection cavity in a subject, the method involving first determining a size of the SCRC, then administering a dosimetric dose of a radio-labeled antibody into the region of interest, then measuring detected radiation from the region of interest at various times subsequent to administering the dosimetric dose, then determining a residence time based on the measured size of the SCRC plus the measured detected radiation from the region of interest; and, finally, calculating the amount of radio-labeled antibody needed to be administered (the “radio-immunotherapeutic,” or “RIT,”) in order to achieve a predetermined radiation absorbed dose (“RAD”).

A further aspect of this invention relates to a method for dosimetry further involving a computer readable medium having computer executable instructions for calculating the required RIT dose (administered activity), the computer executable instructions for receiving a number of parameters necessary to calculate the SCRC percent-injected dose, the SCRC residence time and the S-value for the SCRC for a given session, and then plugging the parameters into predefined formulas to calculate the required RIT dose (administered activity) and outputting the calculated required RIT dose (administered activity) to a user display. A related aspect of this invention provides computer executable instructions for performing the step of storing the parameters and/or the calculated required RIT dose (administered activity) in a storage device.

A further aspect of this invention relates to a computer implemented method for dosimetry estimation for a region of interest at or around an SCRC in a subject, involving a first step of receiving data representative of the size of a surgically created resection cavity, followed by administering a small “dosimetric dose” of radio-labeled antibodies directly into the SCRC or the surrounding area, and detecting the amount of radiation remaining at various time points after administration of this dosimetric dose. Then, using the size of the SCRC and the amounts of radiation measured after administration of the dosimetric dose, the residence time (i.e., how long the radio-labeled antibody remains in the body after administration) is calculated. Finally, based on the size of the SCRC and the residence time of the radio-label, the amount of radioactivity (labeled or conjugated to anantibody) needed to be administered (the “radio-imnmunotherapeutic,” or “RIT” dose) in order to achieve a predetermined radiation absorbed dose (“RAD”) (this is the dose of radiation that needs to be absorbed by the diseased or cancerous tissue in order for the therapy to be effective) is calculated.

Another aspect of this invention relates to a method of enhancing delivery of therapeutic antibodies that specifically bind to an extracellular stromal constituent of a tumor in a mammalian subject, involving administering to a subject in need thereof an effective dosage of unlabeled blocking antibodies, where the unlabeled blocking antibodies specifically binding to substantially all of non-target extracellular stromal constituent of normal tissue while binding to a substantially small percentage of the extracellular stromal constituent of said tumor, thus blocking the binding of therapeutic antibodies to non-target extracellular stromal constituent of normal tissue; and then administering to the subject a treatment effective amount of therapeutic antibodies, the therapeutic antibodies being specific for the extracellular stromal constituent of the tumor.

Further aspects of this invention relate to the method of enhancing delivery of therapeutic antibodies that specifically bind to an extracellular stromal constituent of a tumor in a mammalian subject, wherein the blocking antibodies are monoclonal antibodies, and/or wherein the therapeutic antibodies are monoclonal antibodies, or wherein therapeutic antibodies are coupled to a therapeutic agent, including, but not limited to, radionuclides, chemotherapeutic agents, and cytotoxic agents.

Further aspects of this invention relate to the method of enhancing delivery of therapeutic antibodies that specifically bind to an extracellular stromal constituent of a tumor in a mammalian subject, wherein the RIT antibodies are conjugated to a radionuclide, and wherein the radionuclide is ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ⁹⁸Au, ¹⁶⁶Ho, ¹¹³mIn, ¹¹⁵mIn, ¹²³I, ¹²⁵I,¹³¹I, ¹⁸⁹Ir, 191Ir, 192Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰y, ²¹²Bi, ¹¹⁹Sb, ¹⁹⁷Hg, ⁹⁷Ru, ¹⁰⁰Pd, ¹⁰¹mRh, or ²¹²Pb.

Further aspects of this invention relate to the method of enhancing delivery of therapeutic antibodies that specifically bind to an extracellular stromal constituent of a tumor in a mammalian subject, wherein the therapeutic antibodies are conjugated to a chemotherapeutic agent, and wherein the chemotherapeutic agent is methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, or cytarabine.

Further aspects of this invention relate to the method of enhancing delivery of therapeutic antibodies that specifically bind to an extracellular stromal constituent of a tumor in a mammalian subject, wherein the therapeutic antibodies are conjugated to a cytotoxic agent, and wherein the cytotoxic agent is ricin, aclacinomycin, diphtheria toxin. Monensin, Verrucarin A, Abrin, Vinca alkaloids, Tricothecenes, and Pseudomonas exotoxin A27.

A further aspect of this invention relates to the method of enhancing delivery of therapeutic antibodies that specifically bind to an extracellular stromal constituent of a tumor in a mammalian subject, wherein the extracellular stromal constituent is fibrinogen, fibronectin, collagen, laminin, proteoglycan, tenascin, entactin, or thrombospondin.

Another aspect of this invention relates to the method of enhancing delivery of therapeutic antibodies that specifically bind to an extracellular stromal constituent of a tumor in a mammalian subject, wherein the administration of the therapeutic antibodies is carried out by intravenous injection. A further aspect of this invention relate to the method of enhancing delivery of therapeutic antibodies that specifically bind to an extracellular stromal constituent of a tumor in a mammalian subject, wherein the therapeutic antibodies are administered by injection. Further aspects of this invention relate to the method of enhancing delivery of therapeutic antibodies that specifically bind to an extracellular stromal constituent of a tumor in a mammalian subject, wherein the therapeutic antibodies are administered at least 2 days after the blocking antibodies are administered, and more particularly at least 4 days after the blocking antibodies are administered.

Further aspects of this invention relate to the method of enhancing delivery of therapeutic antibodies that specifically bind to an extracellular stromal constituent of a tumor in a mammalian subject, wherein the subject is afflicted with lymphoma, or wherein the subject is afflicted with a brain tumor.

A further aspect of this invention relate to the method of enhancing delivery of therapeutic antibodies that specifically bind to an extracellular stromal constituent of a tumor in a mammalian subject, wherein each subject is monitored for an adverse reaction to the blocking antibodies administered in the first step, and further where subjects who experience such an adverse reaction the blocking antibodies administered in the first step do not receive an administration of therapeutic antibodies at the second step.

A further aspect of this invention relates to the use of an unlabeled blocking antibody for the preparation of a medicament for use in a method of enhancing delivery of therapeutic antibodies that specifically bind to an extracellular stromal constituent of a tumor in a mammalian subject, involving administering to a subject in need thereof an effective dosage of unlabeled blocking antibodies, where the unlabeled blocking antibodies specifically binding to substantially all of non-target extracellular stromal constituent of normal tissue while binding to a substantially small percentage of the extracellular stromal constituent of said tumor, thus blocking the binding of therapeutic antibodies to non-target extracellular stromal constituent of normal tissue; and then administering to the subject a treatment effective amount of therapeutic antibodies, the therapeutic antibodies being specific for the extracellular stromal constituent of the tumor.

### BRIEF DESCRIPTION OF THE DRAWINGS

FIG. 1 is a schematic diagram of a data processing system according to embodiments of the present invention. FIG. 1 depicts a data processing system 5 that includes a processor 10 that communicates with a memory 14 over a data bus 48. The memory 14 may include several categories of software and data used in the data processing system 5: the operating system 60; the application programs 54; the input/output (I/O) device drivers 58 and the data 56. The data 56 may include magnetic resonance imaging (MRI) data 50, which may be obtained from an MRI system 25 that is configured to provide an image of a subject, for example to determine a resection cavity size. The data 56 may also include scintigraphy data 52, which may be obtained from a scintigraphy system 20 that is configured to detect radiation, for example, using scintigraphy techniques known to those of skill in the art. The application programs 54 can include an administered dose module 10 configured to calculate an administered therapeutic RIT dose, for example, using the techniques described herein. The administered dose module 10 can estimate dosimetry for a region of interest at or around a resection cavity in a subject.

FIG. 2 is a is a flowchart illustrating dosimetry operations according to the embodiments of the present invention. FIG. 2 depicts the following steps: 1) determination of the resection cavity (or SCRC) size (Block 102); 2) determination of the residence time (Block 104); and 3) calculation of the administered RIT dose (Block 106).

### DETAILED DESCRIPTION

### Definitions

“Dosimetry” as used herein refers to the accurate measurement and determination of dosages, especially radiation dosages.

“Magnetic Resonance Imaging” (MRI) as used herein is a radiology technique designed to image internal structures of the body using magnetism, radio waves, and a computer to produce the images of body structures. In magnetic resonance imaging (MRI), the scanner is a tube surrounded by a giant circular magnet. The patient is placed on a moveable bed that is inserted into the magnet. The magnet creates a strong magnetic field that aligns the protons of hydrogen atoms, which are then exposed to a beam of radio waves. This spins the various protons of the body, and they produce a faint signal that is detected by the receiver portion of the MRI scanner. A computer processes the receiver information, and an image is produced. The image and resolution is quite detailed and can detect tiny changes of structures within the body, particularly in the soft tissue, brain and spinal cord, abdomen and joints. MRI is well known in the art.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. For example, in the field of radiology, “about 44 Gy” can mean 44±20% (a range of 35.2 to 52.8 Gy), due to inherent difficulties in measuring radiation absorbed dose accurately. Preferably, in practice, “about 44 Gy” means 44 Gy±10% (a range of 39.6 to 48.4 Gy), but this level of accuracy may be difficult to achieve. The term “pharmaceutically acceptable” as used herein means biologically or pharmacologically compatible for in vivo use in animals or humans, and preferably means approved by a regulatory agency of the Federal or a state government or listed in the U.S. Pharmacopoeia or other generally recognized pharmacopoeia for use in animals, and more particularly in humans.

A “surgically created resection cavity” (“SCRC”) is a cavity in the brain that is surgically created during the removal of a brain tumor, such as a glioblastoma multiforme (“GBM”). A “margin” is a region of parenchyma or brain tissue surrounding the SCRC and may be expressed in terms of a distance from the SCRC/parenchyma interface, or outside edge of the SCRC.

A “region of interest” (“ROI”) as used herein is a defined region of tissue in or near the SCRC. An ROI may be a region that is located at the margin (or outside edge) of the SCRC. In one embodiment, the ROI is a 5 cm wide area of parenchymal tissue surrounding and encircling the SCRC, extending away from the margin (or outside edge) of the SCRC. In another embodiment, the ROI is a 4 cm wide area of parenchymal tissue surrounding and encircling the SCRC, extending away from the margin (or outside edge) of the SCRC. In another embodiment, the ROI is a 3 cm wide area of parenchymal tissue surrounding and encircling the SCRC, extending away from the margin (or outside edge) of the SCRC. In another embodiment, the ROI is a 2 cm wide area of parenchymal tissue surrounding and encircling the SCRC, extending away from the margin (or outside edge) of the SCRC. In another embodiment, the ROI is a 1 cm wide area of parenchymal tissue surrounding and encircling the SCRC, extending away from the margin (or outside edge) of the SCRC. In another embodiment, the ROI is a 0.5 cm wide area of parenchymal tissue surrounding and encircling the SCRC, extending away from the margin (or outside edge) of the SCRC. Alternatively, an ROI may be any-sized area surrounding and encircling the SCRC, as needed. “Parenchyma,” or “parenchymal tissue” as used herein consists of tissue composed of functional cells. Parenchymal cells are much less tolerant of a degraded environment, e.g., an SCRC, than are structural cells, or mesenchymal tissue. An SCRC “interface:” as used herein describes the border between the SCRC and surrounding healthy tissue.

A “residence time” as used herein is a measurement of how long a radionuclide is retained in the body. A “whole-body clearance rate” is a total body residence time.

An “S-value” as used herein is a value that describes the absorbed dose to a specific target region from radiation emitting from another source. S-values may be derived using Monte Carlo methods and MIRD phantom, according to calculations known by those of skill in the art. S-values are dependent on the size of the surgically created resection cavity (SCRC), which may range from about 2 cm³ (an S-value of about 9.60E-3 Gy hr mCi⁻¹) all the way up to about 60 cm³ (an S-value of about (2.34E-3 Gy hr mCi⁻¹), or beyond.

An “absorbed dose” as used herein is the radiation energy (or radioactivity)absorbed (or “deposited”) in a region of interest or other material per unit of mass of the material. This is different from the “administered” or “therapeutic” or “radioimmunotherapy” (RIT) dose, which is the total amount of radioactivity administered to a subject. The absorbed dose refers only to the amount of radiation energy (or radioactivity) that has been administered and absorbed (or “deposited”) into tissue. “Absorbed dose” is alternatively known as “Radiation Absorbed Dose,” or “RAD.” If the absorbed dose or RIT dose is determined before employing the dosimetric methods according to this invention to estimate an RIT dose, the absorbed dose or RAD is called a “predetermined absorbed dose” or a “predetermined RAD.” The predetermined RAD may be a predetermined optimal RAD based on experimental data. A predetermined optimal RAD may be determined by any means accepted in the field of cancer or disease therapy, including experimental trials where the safety and efficacy of a particular radiotherapeutic agent can be determined. For example, an optimal RAD can be determined based on toxicity and clinical outcome in an observed group of subjects. In one embodiment, the predetermined optimal RAD is about 44 Gy.

A “radioimmunotherapy dose” (“RIT dose”) as used herein is the dose of an RIT agent to be delivered for therapeutic purposes. A therapeutic RIT dose is calculated to achieve a predetermined radiation absorbed dose (RAD).

A “targeting moiety” as used herein is any moiety that is able to bind to, i.e., a “binding partner of,” the intended target of the therapy, and deliver an amount of a radio-label (radiotherapeutic agent), chemotherapeutic agent, cytotoxic agent, or other therapeutic agent known in the art. For instance, a targeting moiety may be a receptor ligand in instances when the target is a cellular receptor. Preferably, therapy agent is an antibody, e.g., 81 C6 monoclonal antibody. When the targeting moiety is an antibody and the therapeutic agent is a radio-label, the complex may be called a radioimmunotherapy (RIT) dose.

A “dosimetric dose” as described herein is a small dose (“sub-therapeutic”) used to calculate a RIT dose to be administered in the future. A number of dosimetric doses are administered in increasing amounts, after which a series of dose-response analyses are performed and the desired RIT dose is determined, based on a predetermined absorbed dose.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ¹¹³mIn, ¹¹⁵mIn, ¹²³I, ¹²⁵I, ¹³¹I, ¹⁸⁹Ir, ¹⁹¹Ir, ¹⁹²Ir, ¹⁹⁴Ir, ⁵²Fe, ⁵⁹Fe, ⁵⁹Fe, ¹⁷⁷Lu, ¹⁰⁹Pd,³²P, ²²⁶ Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta ¹¹⁷mSn, ¹²¹Sn, ¹⁶⁶Yb, ¹⁶⁹Yb, ⁹⁰Y, ²¹²Bi, ¹⁹Sb, ⁹⁷Hg, ⁹⁷Ru, ¹⁰⁰Pd, ¹⁰¹mrh, and ²¹²Pb.

“Antibody” or “antibodies” as used herein refers to all types of immunoglobulins, including IgG, IgM, IgA, IgD, and IgE. The term “immunoglobulin” includes the subtypes of these immunoglobulins, such as IgG1, IgG2, IgG3, IgG4, etc. Of these immunoglobulins, IgM and IgG are preferred, and IgG is particularly preferred. The antibodies may be of any species of origin, including (for example) mouse, rat, rabbit, horse, or human, or may be chimeric antibodies. The term “antibody” as used herein includes antibody fragments which retain the capability of binding to a target antigen, for example, Fab, F(ab′)2, and Fv fragments, single-chain antibodies (“scAbs”), and the corresponding fragments obtained from antibodies other than IgG. Such fragments are also produced by techniques that are well known in the art.

“Extracellular stromal constituent” as used herein refers to a compound specific to the extracellular (as opposed to the cellular or cell surface) space, including the glycocalyx, the extracellular matrix, and the basal lamina. Examples of extracellular stromal constituents include but are not limited to fibrinogen, fibronectin, collagen, laminin, proteoglycan, tenascin, entactin, and thrombospondin. If the cellular constituent comprises tumor or cancer cells, the extracellular stromal constituent is the extracellular stromal constituent “of the tumor.” A blocking antibody that binds tenascin in the extracellular stromal constituent will bind to tenascin molecules in the extracellular stromal constituent of both normal and tumorous tissue.

“Chemotherapeutic agent” as used herein includes but is not limited to methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine

“Cytotoxic agent” as used herein includes but is not limited to ricin (or more particularly the ricin A chain), aclacinomycin, diphtheria toxin. Monensin, Verrucarin A, Abrin, Vinca alkaloids, Tricothecenes, and Pseudomonas exotoxin A.

“Radioimmunotherapy” or “RIT” as used herein refers to therapy using an antibody conjugated to a radionuclide (or radio-label).

“Chemoimmunotherapy” as used herein refers to therapy using an antibody conjugated to a chemotherapeutic agent.

“Cytotoxic immunotherapy” as used herein refers to therapy using an antibody conjugated to a cytotoxic agent.

A “therapeutic antibody” as used herein is an antibody that is conjugated to a radionuclide (or “radio-label”), a chemotherapeutic agent, or a cytotoxic agent. When the therapeutic antibody is conjugated to a radionuclide (or radio-label), it is known as an RIT agent or an RIT antibody.

An “unlabeled blocking antibody” as used herein describes an antibody that is not conjugated to a radionuclide (or radio-label), a chemotherapeutic agent, or a cytotoxic agent. When the corresponding therapeutic antibody is conjugated to a radionuclide (or radio-label), the unlabeled blocking antibody is also known as a “cold” antibody.

A “a substantially small percentage” as used herein means less than or equal to 20%, preferably less than or equal to 10%, more preferably less than or equal to 5%, and ideally less than or equal to 1%.

A “therapeutically effective amount” as used herein means the amount of a compound that, when administered to a subject for treating a state, disorder or condition is sufficient to effect a treatment (as defined below). The “therapeutically effective amount” will vary depending on the compound, the disease and its severity and the age, weight, physical condition and responsiveness of the subject to be treated. According to the present invention, in one embodiment, a therapeutically effective amount of radio-labeled antibody is an amount effective to treat various cancers. In another embodiment, a therapeutically effective amount of unlabeled antibody is an amount effective to block the binding of radio-labeled antibody to healthy, non-target tissue.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms.” to any type of treatment that imparts a benefit to a subject afflicted with a disease, including improvement in the condition of the subject (e.g., in one or more symptoms), delay in the progression of the disease, etc.

“Treatment effective amount” as used herein means an amount of the antibody sufficient to produce a desirable effect upon a subject inflicted with lymphoma, including improvement in the condition of the subject (e.g.,, in one or more symptoms), delay in the progression of the disease, etc.

A “subject” or “subject in need” is a human or non-human mammal in need of radioimmunotherapy” (RIT), chemoimmunotherapy, cytotoxic immunotherapy or some other therapeutic method according to the present invention.

### Antibodies used in the Treatment of Cancer

Antibodies according to this invention are any antibodies that are targeted to proteins that are both overexpressed and secreted from the expressing cell into the surrounding environment, such as the extracellular matrix or the extracellular stroma. Such antibodies may be polyclonal (i.e., serum or an antibody preparation purified from serum; “PAb”) or monoclonal (“MAb”). Such monoclonal antibodies include, but are not limited to, monoclonal antibody 81C6. Antibodies according to this invention include antibodies from any species, including, but not limited to, human, mouse (murine), rabbit, goat and pig antibodies. Such antibodies include, but are not limited to, murine monoclonal antibody 81 C6 (m81 C6). Antibodies according to this invention may also be from multiple species (i.e., chimeric). Such antibodies include, but are not limited to, human/mouse chimeric monoclonal antibody 81 C6 (ch8 1 C6), which contains mouse 81 C6 variable regions and human IgG2 constant regions. Antibodies that bind to extracellular stromal constituents of cancers and tumors are known and described in, for example, U.S. Pat. Nos. 6,783,760 and 6,749,853.

Antibodies employed in carrying out the present invention are those that bind to tenascin. Particularly preferred are monoclonal antibody 81 C6 and antibodies that bind at or near the epitope bound by monoclonal antibody 81C6 (i.e., antibodies that cross-react with, or block the binding of, monoclonal antibody 81C6). The monoclonal antibody 81C6 is a murine IgG2b monoclonal antibody raised from a hybridoma fusion following immunization of BALB/c mice with the glial fibrillary acidic protein (GFAP)-expressing permanent human glioma line U-251 MG, as known and described in M. Bourdon et al., Cancer Res. 43:2796-2805 (1983).

Particularly preferred for carrying out the present invention is a mouse-human chimeric monoclonal antibody 81C6, as described in U.S. Pat. No. 5,624,659 (to Bigner and Zalutsky). Antibodies for use in the present invention specifically bind to tenascin with a relatively high binding affinity, for example, with a dissociation constant of about 10⁻⁴ to about 10⁻¹³. In particular embodiments of the present invention, the dissociation constant of the antibody-tenascin complex is at least 10⁻⁴, preferably at least 10⁻⁶, and more preferably at least 10⁻⁹.

The monoclonal antibodies of the present invention may be recombinant monoclonal antibodies produced according to the methods disclosed in U.S. Pat. No. 4,474,893 (to Reading), or U.S. Pat. No. 4,816,567 (to Cabilly et al.). The antibodies may also be chemically constructed by specific antibodies made according to the method disclosed in U.S. Pat. No. 4,676,980 (to Segel et al.). The disclosure of all U.S. patent references cited herein are hereby incorporated in their entirety.

Monoclonal antibodies may be chimeric antibodies produced in accordance with known techniques. For example, chimeric monoclonal antibodies may be complementarily determining region-grafted antibodies (or “CDR-grafted antibodies”) produced in accordance with known techniques.

Monoclonal Fab fragments may be produced in Escherichia coli by recombinant techniques known to those skilled in the art. See, e.g., W. Huse, Science 246, 1275-81 (1989). The antibodies employed in carrying out the present invention are any antibodies that are specific for (bind to) any protein or other macromolecule that is overexpressed to a cancerous or tumorous condition in a subject. In one embodiment of the present invention, the antibodies are those which bind to tenascin. In other embodiments of the present invention, the antibody can be monoclonal antibody

81C6 or an antibody that binds to the epitope bound by monoclonal antibody 81C6 (i.e., antibodies that cross-react with, or block the binding of, monoclonal antibody 81C6). The monoclonal antibody 81C6 is a murine IgG2b monoclonal antibody raised from a hybridoma fusion following immunization of BALB/c mice with the glial fibrillary acidic protein (GFAP)-expressing permanent human glioma line U-251 MG, as known and described in M. Bourdon et al., Cancer Res. 43, 2796 (1983). In other embodiments of the present invention, the antibody can be a polyclonal antibody against a spliced variant of tenascin.

Particularly preferred for carrying out the present invention is a mouse-human chimeric monoclonal antibody 81C6, as described in U.S. Pat. No. 5,624,659 to Bigner and Zalutsky, or a rabbit polyclonal antibody, anti-TNff C-D, as further described in the Examples below. Antibodies for use in the present invention specifically bind to tenascin with a relatively high binding affinity, for example, with a dissociation constant of about 10⁻⁴ to 10⁻¹³. In embodiments of the invention, the dissociation constant of the antibody-tenascin complex is at least 10⁻⁴, preferably at least 10⁻⁶, and more preferably at least 10⁻⁹.

Antibodies of the present invention may be coupled to a radioisotope. The antibody can be coupled to a radioisotope using the techniques described in Current Protocols in Immunology, Volumes 1 and 2, Coligen et al., Ed. Wiley-Interscience, New York, N.Y., Pubs. (1991). Examples of radioisotopes which may be coupled to the antibody include, but are not limited to, 227Ac, 211At, 131Ba, 77Br, 14C, 109Cd, 51Cr, 67Cu, 165Dy, 155Eu, 153 Gd, 198Au, 3H, 166Ho, 113mIn, 115mIn, 123I, 125I, 131I, 189Ir, 191Ir, 192Ir, 194Ir, 52Fe, 55Fe, 59Fe, 177Lu, 109Pd, 32P, 226Ra, 186Re, 188Re, 153Sm, 46Sc, 47Sc, 72Se, 75Se, 105Ag, 89Sr, 35S, 177Ta, 117mSn, 121 Sn, 166Yb, 169Yb, 90Yt, 212Bi, 119Sb, 197Hg, 97Ru, 100Pd, 101mRh, and 212Pb.

It will be appreciated that monoclonal antibodies as used herein incorporate those portions of the constant region of an antibody necessary to evoke the useful immunological response in the subject being affected.

Examples of tumors, cancers, and neoplastic tissue that can be detected and/or diagnosed according to the present invention include, but are not limited to, malignant disorders such as breast cancers; osteosarcomas; angiosarcomas; fibrosarcomas and other sarcomas; leukemias; lymphomas (Hodgkin's lymphoma and Non-Hodgkin's lymphoma), and other blood cancers; myelodysplasia, myeloproliferative disorders; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central nervous system (CNS) and peripheral nervous system (PNS) tumors, malignant or benign, including gliomas and neuroblastomas.

### Subjects

Subjects in need of treatment by the methods described herein include subjects afflicted with malignant disorders, including, but not limited to, breast cancers; osteosarcomas; angiosarcomas; fibrosarcomas and other sarcomas; leukemias; lymphomas (Hodgkin's lymphoma and Non-Hodgkin's lymphoma), and other blood cancers; myelodysplasia, myeloproliferative disorders; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central nervous system (CNS) and peripheral nervous system (PNS) tumors, malignant or benign, including gliomas and neuroblastomas.

Lymphomas that may be treated by methods of the present invention include, but are not limited to, both Hodgkin's lymphoma and non-Hodgkin's lymphoma.

Brain tumors or cancers that may be treated by methods of the present invention include, but are not limited to, glioblastoma multiforme (GBM) and cystic astrocytoma.

### Administration

The labeled and unlabeled antibodies according to this invention may be administered by any medically appropriate procedure known in the art. Such antibodies include those administered as dosimetry dosing antibodies, radioimmunotherapy (RIT) antibodies, unlabeled blocking antibodies, and any labeled therapeutic antibodies for use according to this invention. Routes of administration include, but are not limited to, any needle-facilitated injection, e.g., intravenous (i.v.) injection, intramuscular (i.m.) injection, subcutaneous (s.c.) injection, intra-arterial injection, injection into the cerebrospinal fluid, intra-cavity injection, intrathecal injection, intradermal injection, direct injection into a tumor, or direct injection into any region of interest. Also included is administration through a reservoir implantation, including, but not limited to, a Rickman reservoir implant.

### Formulations

The dosimetric dosing antibodies, radioimmunotherapeutic armintibodies, unlabeled blocking antibodies and other labeled therapeutic antibodies may be mixed, prior to administration, with a non-toxic, pharmaceutically acceptable carrier substance (e.g. normal saline or phosphate-buffered saline), and will be administered using any medically appropriate procedure, e.g. parenteral administration (e.g. , injection) such as by intravenous or intra-arterial injection.

The blocking antibodies and therapeutic antibodies compounds described above may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the subject. The carrier may be a liquid and is preferably formulated with the compound as a unit-dose formulation which may contain from 0.01 or 0.5% to 95% or 99% by weight of the active compound.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient.

Dosimetric dosing, blocking and all therapeutic antibodies according to this invention may be provided in lyophilized form in a sterile aseptic container or may be provided in a pharmaceutical formulation in combination with a pharmaceutically acceptable carrier, such as sterile pyrogen-free water or sterile pyrogen-free physiological saline solution.

In further embodiments, the therapeutic antibodies may optionally be administered in conjunction with other, different, active compounds useful in the treatment of the disorders or conditions described herein (e.g., chemotherapeutics). The other compounds may be administered concurrently. As used herein, the word “concurrently” means sufficiently close in time to produce a combined effect (that is, concurrently may be simultaneously, or it may be two or more administrations occurring before or after each other).

### Dosimetry Estimation of Rit Dose

Dosimetry refers to the accurate measurement of dosages. If the doses in question are radioactivity doses, dosimetry refers to the accurate measurement of the amount of radiation energy in a tissue. This is especially critical when using radiation to treat a subject for a disease or cancer. Thus, using too much radiation is very toxic to the subject, while using too little is ineffective as therapy.

The method according to this invention provides for an accurate determination of a safe and effective RIT dose (the amount of administered radioactivity). First, a so-called “radiation absorbed dose” or “RAD” is determined. This may be done by any means accepted in the field of cancer or disease therapy, including experimental trials where the safety and efficacy of a particular radiotherapeutic agent can be determined. For example, an optimal RAD can be determined based on toxicity and clinical outcome in an observed group of subjects. The amount of the predetermined RAD may be determined to be an amount from about 10 Gy up to about 100 Gy. More preferably, the amount of the predetermined RAD may be determined to be an amount from about 25 Gy up to about 75 Gy. More preferably, the amount of the predetermined RAD may be determined to be an amount from about 30 Gy up to about 60 Gy. More preferably, the amount of the predetermined RAD may be determined to be an amount from about 40 Gy up to about 50 Gy. More preferably, the amount of the predetermined RAD may be determined to be an amount from about 42 Gy up to about 48 Gy. In a preferred embodiment, the amount of the predetermined RAD is about 44 Gy.

Once the predetermined RAD is set, the methods according to this invention may be used to determine what RIT dose should be administered in order to achieve the predetermined RAD. First, a so-called “dosimetric dose” of radio-labeled antibody is administered to a subject in need of radioimmunotherapy, in escalating amounts. In one embodiment, for example, the radio-labeled antibody (or “RIT agent”) is ¹³¹I-labeled 81C6 monoclonal antibody. When using this RIT agent, the dosimetric dose can be between about 1 to 2 mg of 81C6, or up to 5 mCi of ¹³¹I.

Once the dosimetric doses have been administered, a series of dose response readings are taken and used to determine the residence time. Then, an S-value is calculated using methods well known in the art, including, but not limited to, Monte Carlo methods and MIRD phantom.

The values for the predetermined RAD, SCRC size, residence time and S-value are plugged into the following equation and the equation is solved for ^{A}^{₀}, which is the administered dose, or RIT dose needed to achieve the predetermined absorbed dose (RAD):
D_{SCRC}=A₀S(B_{2-cm}←SCRC)τ_{SCRC}
where D^{_{SCRC}} is the predetermined absorbed dose (or RAD), A^{₀} is the administered dose (or RIT dose) expressed in mCi, S(B_{2-cm}←SCRC) is the corresponding S-value, expressed in Gy hr mCi⁻¹, and τ_{SCRC} the residence time in hr.

The therapeutic doses may range from about 10 mCi to about 200 mCi. However, the therapeutic doses are generally between about 20 mCi to about 180 mCi.

Accordingly, a dosimetric dose can be used to calculate the RIT dose needed to achieve a desired or predetermined radiation absorbed dose (RAD) in a subject. Because the RIT dose can be based on a subject's specific residence time as determined by scintigraphy data and the size of the resection cavity, a therapeutic dose can be calculated in order to result in a desired or predetermined absorbed dose in the subject. Therefore, the accuracy of dosing may be increased, and toxic effects of RIT may be decreased.

In another embodiment, whole-body scintigraphy is performed to detect radiation from the region of interest. The region of interest may be a predefined region that includes the SCRC, or may be the parenchyma of the brain that is a predetermined distance from the margin of the SCRC, such as between 1 to 2 centimeters from the margin of the SCRC. The scintigraphy may be performed at a plurality of times, such as a first time that is substantially the same time as the administration of the dosimetric RIT dose, at a second time that is about twenty-four hours subsequent to the first time, and at a third time that is about forty-eight hours subsequent to the first time. Other scintigraphy schedules may be used as needed. For instance, in another embodiment, the scintigraphy is performed at a first time that is substantially the same time as the administration of the dosimetric RIT dose, at a second time that is about 2 to 3 days subsequent to the first time, and at a third time that is about 5 to 7 days subsequent to the first time.

It should be understood that various RIT agents can be used according to embodiments of the present invention. Moreover, the dosimetric RIT dose may be obtained from the same or different RIT agents than the therapeutic RIT dose. For example, an amount of ¹³¹Iodine-labeled anti-tenascin murine 81C6 monoclonal antibody may be used as a RIT agent. Any suitable RIT agent may be used. Other RIT agents may include any suitable radionuclide, for example, ²¹¹Astatine, ¹⁷⁷Lutetiam, and ⁹⁰Yttrium.

The dosimetric dose and/or the therapeutic RIT dose can be administered by any means well known in the art, including, but not limited to, through a reservoir implantation, such as a Rickman reservoir implant.

### Computer-Driven Dosimetry Methods

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Dose data used in subject dose verification can be presented in one of three formats: (a) on a display on the reading instrument, (b) on a print-out from the electronic reader or (c) on a computer screen. In the latter case, the information presented on the computer screen is in the form of numbers or tables and, in some cases, graphs.

The above described computer and computer software can be used to calculate the required administered activity to deliver a given dose to a specifically defined cavity margin. The computer software can make such calculations using data input from a reading instrument or the user, as well as, any calibration or correction factors previously input by the user, typically following a previous calibration of the dosimetry system in a known manner. The software may then compare the dose calculations with a predetermined target doses and indicate, conveniently by highlighting in the display, any deviation for corrective action. Furthermore, the software can manipulate and apply any formulas discussed herein to all data input into and received by the computer. The computer can create and store tables and accurately apply all input data into the formulas and output the required administered activity A0 to deliver a specific target dose to a pre-determined cavity margin.

The above computer software implementation can be accomplished by providing a computer readable medium having computer executable instructions for calculating the required administered activity. The computer executable instructions are configured to receive a number of parameters necessary to calculate the SCRC percent-injected dose, the SCRC residence time and the S-value for the SCRC for a given session. Once the parameters are received the computer executable instructions will plug the parameters into predefined formulas to calculate the required administered activity. The required administered activity and any of the input parameters can then be output to a user display for analysis. Also useful, the executable instructions can be set up to store the parameters in a storage device.

Additionally, as briefly discussed above, with the aid of a computer, a medical practitioner (e.g., a radiation oncologist or a medical physicist or a physician) can determine the target RIT dose to a high degree of accuracy at a point in the resection cavity. As a criterion for accepting or rejecting a given source configuration, this dose is likely to be applied at the periphery of an implanted tumor, where the concern is to minimize damage to normal tissue. In view of the practical difficulties of source localization, tissue heterogeneity, and measurement accessibility, all of which unavoidably contribute heavily to dosimetric uncertainty in the clinical setting, it is reasonable to require that the component of error associated with predetermined single-source dose distributions be as accurate and reproducible as possible given this aforementioned difficulties.

Although embodiments of the invention are described with respect to the data processing system 5 and the administered dose module 10, it should be understood that other configurations may be used to carry out operations according to embodiments of the invention. For example, the administered dose module 10 may be incorporated into other components of the memory 14, such as the operating system 60. Techniques other than MRI imaging and scintigraphy, as used by one of ordinary skill in the art, may also be used to determine the size of the resection cavity and/or to detect radiation from a region of interest in the subject.

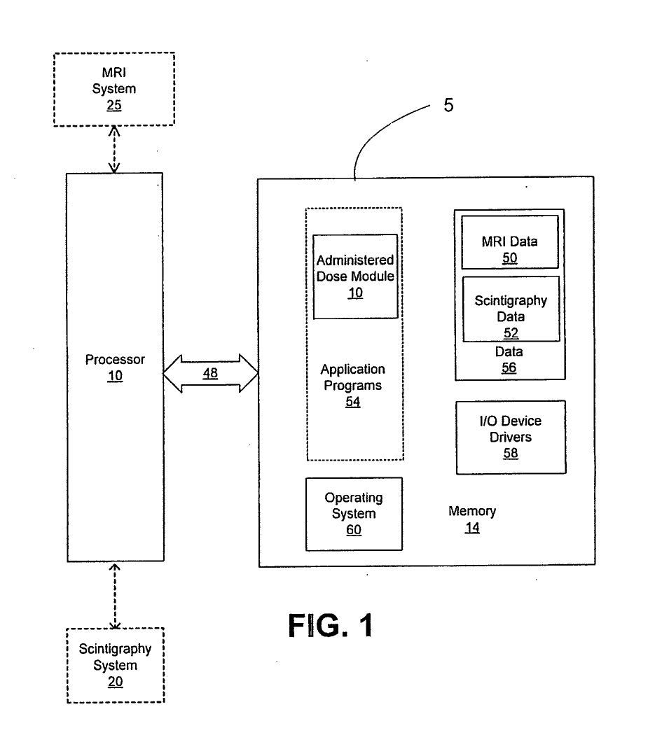

An exemplary system according to embodiments of the present invention is illustrated in FIG. 1. As illustrated, a data processing system 5 includes a processor 10 that communicates with a memory 14 over a data bus 48. The memory 14 may include several categories of software and data used in the data processing system 5: the operating system 60; the application programs 54; the input/output (I/O) device drivers 58 and the data 56.

The data 56 may include magnetic resonance imaging (MRI) data 50, which may be obtained from an MRI system 25 that is configured to provide an image of a subject, for example to determine a resection cavity size. The data 56 may also include scintigraphy data 52, which may be obtained from a scintigraphy system 20 that is configured to detect radiation, for example, using scintigraphy techniques known to those of skill in the art.

The application programs 54 can include an administered dose module 10 configured to calculate an administered therapeutic RIT dose, for example, using the techniques described herein. The administered dose module 10 can estimate dosimetry for a region of interest at or around a resection cavity in a subject.

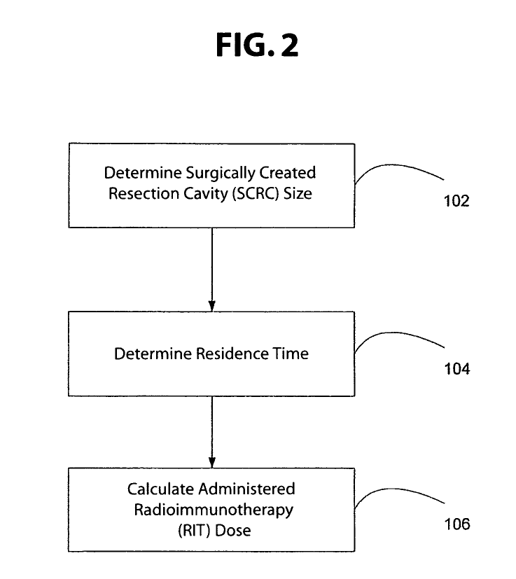

For example, the administered dose module 10 can carry out one or more of the following operations with reference to FIG. 2. The size of the resection cavity can be determined (Block 102), for example, by calculating the size based on an MRI image from the MRI system 25 in FIG. 1. A dosimetric RIT dose can be administered to a resection cavity of the subject, and the size MRI image may be obtained before or after the dosimetric RIT dose is administered. After the dosimetric RIT dose is administered, the residence time may be determined (Block 104). For example, the administered dose module 10 can calculate the residence time based on the size of the cavity and detected radiation from the region of interest at a plurality of times subsequent to administering the dosimetric dose. The detected radiation can be obtained by the administered dose module 10 from scintigraphy data 52 from the scintigraphy system 20. An administered therapeutic RIT dose can be calculated based on the residence time, the size of the resection cavity, and a predetermined absorbed dose (Block 106).

Accordingly, a dosimetric RIT dose can be used to calculate a therapeutic dose for fuiture use to obtain a desired absorbed dose in the subject. Because the therapeutic dose can be based on a subject specific residence time as determined by scintigraphy data and the size of the resection cavity, a therapeutic dose can be calculated in order to result in a desired or predetermined absorbed dose in the subject. Therefore, the accuracy of dosing may be increased, and toxic effects of RIT may be decreased. The predetermined absorbed dose can be an optimal absorbed dose based on experimental data. For example, an optimal dose can be determined based on toxicity and clinical outcome in an observed group of subjects. The amount of the predetermined RAD may be determined to be an amount from about 10 Gy up to about 100 Gy. More preferably, the amount of the predetermined RAD may be determined to be an amount from about 25 Gy up to about 75 Gy. More preferably, the amount of the predetermined RAD may be determined to be an amount from about 30 Gy up to about 60 Gy. More preferably, the amount of the predetermined RAD may be determined to be an amount from about 40 Gy up to about 50 Gy. More preferably, the amount of the predetermined RAD may be determined to be an amount from about 42 Gy up to about 48 Gy. In a preferred embodiment, the amount of the predetermined RAD is about 44 Gy.

Once the predetermined RAD is set, the methods according to this invention may be used to determine what RIT dose should be administered in order to achieve the predetermined RAD. In one embodiment, the predetermined RAD is about 44 Gy. The dosimetric RIT dose can be between about 1 and 2 mg of ¹³¹I-labeled 81C6, or up to about 5 mCi of ¹³¹ I. The therapeutic doses are generally between about 10 mCi to about 200 mCi.

Although embodiments of the invention are described with respect to the data processing system 5 and the administered dose module 10, it should be understood that other configurations may be used to carry out operations according to embodiments of the invention. For example, the administered dose module 10 may be incorporated into other components of the memory 14, such as the operating system 60. Techniques other than MRI imaging and scintigraphy may also be used to determine the size of the resection cavity and/or to detect radiation from a region of interest in the subject.

In one embodiment, whole-body scintigraphy is performed to detect radiation from the region of interest. The region of interest may be a predefined region that includes the parenchyma of the brain that is a predetermined distance from the margin of the resection cavity, such as between 1 to 2 centimeters from the margin of the resection cavity. The scintigraphy may be performed at a plurality of times, such as a first time that is substantially the same time as the administration of the dosimetric RIT dose, at a second time that is about twenty-four hours subsequent to the first time, and at a third time that is about forty-eight hours subsequent to the first time.

It should be understood that various RIT agents can be used according to embodiments of the present invention. Moreover, the dosimetric RIT dose may be obtained from the same or different RIT agents than the therapeutic RIT dose. For example, an amount of ¹³¹Iodine-labeled anti-tenascin murine 81C6 monoclonal antibody may be used as a RIT agent. Any suitable RIT agent may be used. Other RIT agents may include any suitable radionuclide, for example, ²¹¹Astatine, ¹⁷⁷Lutetiam, and ⁹⁰Yttrium. The dosimetric dose and/or the therapeutic RIT dose can be administered through a reservoir implantation, such as a Rickman reservoir implant.

### Blocking Antibodies

Another aspect of the present invention concerns the use of blocking antibodies in methods relating to the treatment of human subjects, including male and female subjects and neonatal, infant, juvenile, adolescent, adult, and geriatric subjects, but the invention may also be carried out on animal subjects, particularly mammalian subjects such as mice, rats, dogs, cats, livestock and horses for veterinary purposes.

In another embodiment, these methods may be used for drug screening and drug development purposes.

1. Blocking Antibody Methods

The blocking antibody methods according to the present invention rely on the phenomenon that certain macromolecules normally present in the body are overexpressed by cancer cells. For example, tenascin is overexpressed by tumor cells associated with brain cancer, lymphoma and other cancers. Therefore, an antibody, e.g., 81C6, that is targeted to tenascin will saturate its specific binding sites (or “epitopes”) near normal healthy cells before it will saturate the binding sites on the tenascin that is overexpressed by cancer cells. The goal of RIT is to bind to and kill only the targeted cancer cells, and leave non-targeted healthy cells alone. This goal can be accomplished by using the methods according to the present invention. For example, a medical practitioner can pre-treat a subject with a sub-therapeutic amount of unlabeled (“cold”) MAb 81C6. The amount of cold antibody administered can be adjusted so that the tenascin molecules expressed by normal, healthy cells are saturated, or nearly saturated, with 81C6 antibodies (i.e., 100% or nearly 100% of the available tenascin molecules expressed by normal, healthy cells are bound by antibody). On the other hand, this small amount of 81C6 will be sufficient to occupy only substantially less of the tenascin molecules that are overexpressed by cancer cells, leaving most of those tenascin molecules unoccupied and thus “susceptible” to subsequent binding by radio-labeled 81C6 antibodies. Preferably, because of the effect of pre-administration of unlabeled blocking antibodies, there is at least a two-fold increase in binding of therapeutic antibodies to the extracellular stromal constituents of a tumor, than there would have been in the absence of blocking antibodies. More preferably, there is at least a five-fold increase in binding of therapeutic antibodies to the extracellular stromal constituents of a tumor, than there would have been in the absence of blocking antibodies. Therefore, pre-treatment with cold antibodies according to the present invention will effectively “shield” or “block” all or nearly all healthy tenascin-producing cells from a subsequent administration of radio-labeled 81C6 antibodies, while at the same time blocking only a small percentage of the tenascin-overexpressing cancer cells from subsequent radioimmunotherapy (“RIT”) with radio-labeled 81C6 antibodies. Furthermore, RIT does not require saturation of the target molecules (tenascin, in this case) to be effective. Thus, after pre-treatment with cold 81C6 antibodies, a therapeutic dose of radio-labeled antibodies can be administered as RIT, and these radio-labeled antibodies will be able to preferentially bind tenascin near the cancer-afflicted cells, killing them.

2. Antibodies and Therapeutic Antibodies.

Blocking antibodies used to carry out the present invention are, in general, not coupled or conjugated to any therapeutic agent, while therapeutic antibodies used to carry out the present invention are, in general, coupled or conjugated to a therapeutic agent. Thus blocking antibodies are not themselves therapeutically active in treating cancer in the methods described herein.

Antibodies used for therapy (i.e., in a method of combating cancer) may be polyclonal or monoclonal antibodies per se or monoclonal antibodies coupled to a therapeutic agent. Such antibodies are sometimes referred to herein as therapeutic antibodies.

Any therapeutic agent conventionally coupled to a monoclonal antibody may be employed, including (but not limited to) radionuclides, cytotoxic agents, and chemotherapeutic agents. See generally Monoclonal Antibodies and Cancer Therapy (R. Reisfeld and S. Sell Eds. 1985)(Alan R. Liss Inc. N.Y.). Therapeutic agents such as radionuclides, cytotoxic agents and chemotherapeutic agents are described above, and also described in U.S. Pat. Nos. 6,787,153; 6,783,760; 6,676,924; 6,455,026; and 6,274,118.

Therapeutic agents may be coupled to the antibody by direct means or indirect means (e.g., via a chelator) by any suitable technique, including but not limited to those described in U.S. Pat. Nos. 6,787,153; 6,783,760; 6,676,924; 6,455,026; and 6,274,118. Therapeutic agents may be coupled or conjugated to the antibody by the Iodogen method (see, e.g., P. J. Fraker and J. C. Speck, Jr., Biochem. Biophys. Res. Commun. 80(4):849-857 (1978)) or with N-succinimidyl-3-(tri-n-butylstanyl)benzoate (the “ATE method”), as will be apparent to those skilled in the art. See, e.g. , M. Zalutsky and A. Narula, Int. J. Rad. Appl. Instrum. 38:1051-1055(1987).

3. Dosage of Blocking and Therapeutic Antibodies.

Dosage of the blocking antibody will depend, among other things, the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher. Dosage of the therapeutic antibody will likewise depend, among other things, the condition of the subject, the particular category or type of cancer being treated, the route of administration, the nature of the therapeutic agent employed, and the sensitivity of the tumor to the particular therapeutic agent. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 0.05, 0.1, 0.5, 1, 5, 10, 20 or 50 micrograms per kilogram subject body weight, or lower, and as high as about 5, 10, 20, 50, 75 or 100 micrograms per kilogram subject body weight, or even higher.

In another example, where the therapeutic agent is ¹³¹I, the dosage to the subject will typically be from 10 mCi to 100, 300 or even 500 mCi. Stated otherwise, where the therapeutic agent is ¹³¹I, the dosage to the subject will typically be from 5,000 Rads to 100,000 Rads (preferably at least 13,000 Rads, or even at least 50,000 Rads). Doses for other radionuclides are typically selected so that the tumoricidal dose will be equivalent to the foregoing range for ¹³¹I, even though the amount of radiation may be different. For example, only a few millicuries of ²¹¹ At may be required to deliver a radiation dose to tumors the equivalent of that delivered by 100 millicuries of ¹³¹I.

In some respects, the administration of blocking antibodies and subsequent administration of therapeutic antibodies can be carried out in like manner as described in U.S. Pat. No. RE38,008 (to Abrams et al.). However, an advantage of the present invention is that blocking antibodies specific for extracellular constituents as described herein have a longer half-life in vivo than do antibodies specific for cells. Hence there may be a longer time period or between administration of the blocking antibody and the subsequent administration of the therapeutic antibody. Thus, in one embodiment, the first dose of the therapeutic antibody is administered to the subject at least one day after the administration of the dose (or last dose, if more than one is given) of the blocking antibodies. In another embodiment, the first dose of the therapeutic antibody is administered to the subject at least two days after the administration of the dose (or last dose, if more than one is given) of the blocking antibodies. In another embodiment, the first dose of the therapeutic antibody is administered to the subject at least three days after the administration of the dose (or last dose, if more than one is given) of the blocking antibodies. In yet another embodiment, the first dose of the therapeutic antibody is administered to the subject at least four days after the administration of the dose (or last dose, if more than one is given) of the blocking antibodies. In another embodiment , the first dose of the therapeutic antibody is administered to the subject at least one week after the administration of the dose (or last dose, if more than one is given) of the blocking antibodies. In another embodiment, the first dose of the therapeutic antibody is administered to the subject at least two weeks after the administration of the dose (or last dose, if more than one is given) of the blocking antibodies. The greater time period between blocking and therapeutic doses that is possible in these embodiments of the present invention advantageously permits greater opportunity for monitoring subject health before administration of the therapeutic antibody (e.g. , observe for toxicity, allergic reaction, anaphylaxis, liver and/or spleen toxicity, etc.). This permits the blocking dose to be administered to the subject on an outsubject basis (which is much less costly, and potentially much more convenient for the subject). This also provides an opportunity, if desired, to avoid administering of the therapeutic antibody if an adverse reaction (e.g., toxicity, allergic reaction, anaphylaxis, liver and/or spleen toxicity, etc., sufficiently serious to discontinue the planned therapeutic treatment) to the administration of the blocking antibody is observed.

### EXAMPLES

The present invention will be better understood by reference to the following Examples, which are provided as exemplary of the invention, and not by way of limitation.

### Example 1

### Dosimetry of ¹³¹I-Labeled Murine 81C6 Monoclonal Antibody for an Absorbed Targeted Dose of 44 Gv

Basic Formulation

According to embodiments of the present invention, dosimetry estimates may be carried out in order to estimate the necessary administered activity ^{A}^{₀} to achieve a targeted dose D of 44 Gy to the 2-cm cavity margins. The basic equation is given by
D_{SCRC}=A₀S(B_{2-cm}←SCRC)τ_{SCRC}
where D_{SCRC} is the targeted dose of 44 Gy, A₀ is the administered activity expressed in mCi, S(B_{2-cm}←SCRC) is the corresponding S-value in Gy hr mCi⁻¹, and τ_{SCRC} the residence time in hr.
Scintigraphy Studies

1. Whole-Body Scintigraphy.

Three or more sessions are used. Each session consists of 1) a whole-body image of the subject, 2) whole-body imaging of the background, and 3) whole-body imaging of a source vial containing initially approximately 200 μCi of 131I. The first session is acquired immediately after the administration of a dosimetric dose of ¹³¹I-labeled murine 81C6 MAb. The second and third is acquired within 24 and 48 hours after administration.

The acquisition parameters:
i. Use of high-energy collimators.
ii. Head speed of 30 cm min .
iii. Maximum length as permitted by the camera.

2. Magnetic Resonance Imaging of the Head.

A MRI of the head is acquired 24 hours prior to the administration of the dosimetric dose. The MRI consists of 3-mm thick, consecutive, zero-spacing, axial T1-weighted contrast-enhanced images. These MRI images are used to assess the volume of the SCRC.

Estimation of the Residence Time in the SCRC

An identical circular region of interest (ROI) is drawn in all planar whole-body images around the SCRC. Total counts from this ROI must be obtained from the anterior and posterior images and from each session, where the percent-injected dose is calculated as follows

$$\% {ID}_{SCRC , i} = \frac{\sqrt{( {CA}_{i}^{SCRC} - {CA}_{i}^{Bkg} ) ( {CP}_{i}^{SCRC} - {CP}_{i}^{Bkg} )}}{\sqrt{( {CA}_{1}^{SCRC} - {CA}_{1}^{Bkg} ) ( {CP}_{1}^{SCRC} - {CP}_{1}^{Bkg} )}} \times 100$$

where % ID_{SCRC,i} is the SCRC percent-injected dose for session i, CA^{SCRC} and CP^{SCRC} represent the anterior and posterior SCRC counts, CA^{Bkg} and CP^{Bkg} represent the anterior and posterior background counts. These calculations are carried out for each session i. The SCRC residence time is calculated using the following formula

$${τ}_{SCRC} = \frac{( \begin{matrix}3 ( {t}_{1}^{2} + {t}_{2}^{2} + {t}_{3}^{2} ) - \\ {( ln ( \% {ID}_{SCRC , 1} ) + ln ( \% {ID}_{SCC , 2} ) + ln ( \% {ID}_{SCRC , 3} ) )}^{2}\end{matrix} )}{3 ( \begin{matrix}( {t}_{1} + {t}_{2} + {t}_{3} ) ( ln ( \% {ID}_{SCRC , 1} ) + ln ( \% {ID}_{SCRC , 2} ) + \\ ln ( \% {ID}_{SCRC , 3} ) ) - {t}_{1} ln ( \% {ID}_{SCRC , 1} ) + \\ {t}_{2} ln ( \% {ID}_{SCRC , 2} ) + {t}_{3} ln ( \% {ID}_{SCRC , 3} ) -\end{matrix} )}$$

The S-Values

S-values for the SCRC are calculated based on cavity size. A table of S-values as a function of cavity volume has been established and is presented in Table 1. Cavity volumes may be interpolated among the values given in the Table 1. The S-values are calculated based on Monte Carlo simulations.

Calculation of the Administered Activity

The required administered activity to deliver a 44 Gy to the 2-cm cavity margins if given by

$${A}_{0} = \frac{{D}_{SCRC}}{S ( {B}_{2 - cm} ← SCRC ) {τ}_{SCRC}}$$

where D_{SCRC} is the target dose of 44 Gy, S(B_{2-cm}←SCRC) is the estimated S-value from Table 1, and τ_{SCRC} is the SCRC residence time.

TABLE 1
SCRC S-values as a function of cavity size

| SCRC Cavity Size (cm3) | S-value (Gy hr mCi⁻¹) |
|---|---|
| 2 | 9.60E−03 |
| 4 | 7.36E−03 |
| 6 | 6.23E−03 |
| 8 | 5.61E−03 |
| 10 | 5.16E−03 |
| 12 | 4.79E−03 |
| 14 | 4.47E−03 |
| 16 | 4.21E−03 |
| 18 | 4.00E−03 |
| 20 | 3.83E−03 |
| 22 | 3.68E−03 |
| 24 | 3.55E−03 |
| 26 | 3.43E−03 |
| 28 | 3.32E−03 |
| 30 | 3.21E−03 |
| 32 | 3.12E−03 |
| 34 | 3.03E−03 |
| 36 | 2.95E−03 |
| 38 | 2.88E−03 |
| 40 | 2.81E−03 |
| 42 | 2.75E−03 |
| 44 | 2.69E−03 |
| 46 | 2.64E−03 |
| 48 | 2.59E−03 |
| 50 | 2.54E−03 |
| 52 | 2.50E−03 |
| 54 | 2.46E−03 |
| 56 | 2.41E−03 |
| 58 | 2.38E−03 |
| 60 | 2.34E−03 |

### Example 2

### Phase II Study of 131-Iodine-Labeled Anti-Tenascin Murine Monoclonal Antibody 81C6 (m81C6) Administered to Deliver a Targeted Radiation Boost Dose of 44 Gy to the Surgically Created Cystic Resection Cavity Perimeter in the Treatment of Subiects with Newly Diagnosed Primary and Metastatic Brain Tumors.

The administration of ¹³¹I-labeled anti-tenascin monoclonal antibody 81C6 (¹³¹I-81C6) into a surgically created resection cavity (SCRC) improves survival for subjects with newly diagnosed and recurrent malignant glioma. Dosimetry analyses from previously-performed studies suggest that the delivery of 44 Gy to the SCRC by ¹³¹I-81C6 is associated with a low rate of toxicity and significant local tumor control. The primary objective of this Example is to evaluate the efficacy and toxicity of administering a dose of ¹³¹I-81C6 to achieve a 44 Gy boost to the SCRC perimeter. Eligibility criteria include: adults with newly diagnosed and previously untreated malignant glioma; gross total resection; no communication between the resection cavity and the cerebrospinal fluid (CSF) space; KPS (Karnofsky performance score; a measure of patient function) greater than 60%; adequate bone marrow, kidney and hepatic function. ¹³¹I-81C6 is administered to achieve a 44 Gy boost to the SCRC based on a pretreatment dosimetry study performed with 5 mCi of ¹²³I-81C6. Conventional external beam radiotherapy and systemic chemotherapy are prescribed to all subjects following ¹³¹I-81C6 administration. Twenty subjects are treated, including 14 with GBM and 6 with AA/AO. The median age is 49.5 years (range, 24 to 70). 67% are male. The median dose of ¹³¹I-81C6 administered is 53 mCi (range, 25 to 150).

Results

All subjects have achieved a 44 Gy (+/−10%) boost to the SCRC perimeter and no subjects have experienced grade 3 or greater toxicity attributable to ¹³¹I-81C6. With a median follow up of 34 weeks, the median survival for all subjects and those with GBM is 94 weeks.

### Example 3

### Dosimetry and Radiographic Analysis of ¹³¹I-Labeled Anti-Tenascin 81C6 Murine Monoclonal Antibody in Newly Diagnosed Subiects with Malignant Gliomas: A Phase II Study

Dosimetry methods according to this invention may be used to estimate the necessary administered activity “AO” as follows: For the purpose of this example, it is assumed that the desired target dose “D” and the size of the cavity margin are known. In the Example, the target RAD (target dose “D”) is 44 Gy and the size of the cavity margin for which the administered radioimmunotherapy dose is being calculated is 2 cm. The S-value can be calculated using Monte Carlo simulations or by alternative means as known in the art. First, the following equation for “Dscrc” is used to determine the value for the administered activity “A0”:
D_{SCRC}=A₀S(B_{2-cm}←SCRC)τ_{SCRC}
where D_{SCRC} is the targeted dose of 44 Gy, A₀ is the administered activity expressed in mCi, S(B_{2-cm}←SCRC) is the corresponding S-value in Gy hr mCi⁻¹, and τ_{SCRC} the residence time in hr., Next, the residence time (τ) in the SCRC is determined. Using the following formula for calculating the residence time (τ), the time (t) of each session (i) is inserted into the formula:

$${τ}_{SCRC} = \frac{( \begin{matrix}3 ( {t}_{1}^{2} + {t}_{2}^{2} + {t}_{3}^{2} ) - \\ {( ln ( \% {ID}_{SCRC , 1} ) + ln ( \% {ID}_{SCC , 2} ) + ln ( \% {ID}_{SCRC , 3} ) )}^{2}\end{matrix} )}{3 ( \begin{matrix}( {t}_{1} + {t}_{2} + {t}_{3} ) ( ln ( \% {ID}_{SCRC , 1} ) + ln ( \% {ID}_{SCRC , 2} ) + \\ ln ( \% {ID}_{SCRC , 3} ) ) - {t}_{1} ln ( \% {ID}_{SCRC , 1} ) + \\ {t}_{2} ln ( \% {ID}_{SCRC , 2} ) + {t}_{3} ln ( \% {ID}_{SCRC , 3} ) -\end{matrix} )}$$

This calculation must be performed for each session (i). Next, the SCRC percent-injected dose “% IDscrc” for each session (i) is calculated using the formula:

$$\% {ID}_{SCRC , i} = \frac{\sqrt{( {CA}_{i}^{SCRC} - {CA}_{i}^{Bkg} ) ( {CP}_{i}^{SCRC} - {CP}_{i}^{Bkg} )}}{\sqrt{( {CA}_{1}^{SCRC} - {CA}_{1}^{Bkg} ) ( {CP}_{1}^{SCRC} - {CP}_{1}^{Bkg} )}} \times 100$$

wherein “CA” and “CP” represent the anterior and posterior SCRC and background radiation counts. Calculation of anterior and posterior SCRC counts is within the ordinary skill in the art and therefore for purposes of this example we will assume this data is readily available or attainable for direct substitution into the formula. Once the SCRC percent-injected dose (% IDscrc) has been calculated for each session (i), these results are substituted directly into the equation for calculating the residence time (r). Multiplying the S-value for the SCRC by the residence time and dividing this product by the target dose D (44 Gy) will yield the required administer activity to deliver 44 Gy to a 2-cm cavity margin.

Changing the size of the cavity margin or target dose in the above example is reflected in the S-value as well as the anterior and posterior SCRC counts. In such a case, the required administered activity can be calculated by recalculating for the S-value and residence time and re-substituting these newly calculated values in the equation for A0 as discussed above.

The following steps are one method of calculating the amount of radiation to be injected in accordance with the present invention. A defined level of radioactivity (mCi 13 I) is injected resulting in a variety of radiation doses (in Gy) received by the 2 cm cavity margin. The cavity size and the cavity residence time is then measured. Then, knowing the mCi injected, the actual radiation dose received by the individual subject can be calculated. Next, looking at the relationship between the radiation dose received by the subject and the outcome, i.e., survival and side effects of the treatment, radiation necrosis, etc., a radiation dose of 44 Gy can be determined to provide the best compromise between maximizing tumor control and minimizing the serious side effect of radiation necrosis.

Accordingly, it is desirable to treat all subjects with a level of radioactivity sufficient to achieve 44 Gy. Knowing that a dose of 44 Gy is desirable we must now calculate how much radioactivity need be injected to achieve 44 Gy. Thus, referring to the equation for AO, D equals 44 Gy, the S-value determines how much energy is deposited in the 2 cm target volume per decay of the I-131. The S-value is geometry dependent and, thus, is a measure of the cavity volume conducted by MRI imaging. The resection cavity residence time reflects how long the radioactivity stays in the cavity, irradiating the target volume, and is measured by serial gamma camera imaging or radiation detector probe measurement.

### Example 4

### Estimation of Maximum Tolerated Dose (MTD) of Unlabeled Human/Mouse Chimeric 81C6 (ch81C6) Antibody in Cancer Subiects and Evaluation of the Pharmacokinetics and Dosimetry of ¹³¹I-Labeled ch81C6 in Cancer Subiects Following Escalated Doses of Unlabeled ch81C6

¹³¹I-labeled chimeric monoclonal antibody 81C6 (¹³¹I-ch81C6) may be safely given to humans intravenously. However, ¹³¹I-ch81C6 binds to tenascin in normal, healthy stroma and in tumorous stroma with equal affinity. Pretreatment of subjects with an unlabeled dose of ch81C6 according to the present invention enhances delivery of later-administered therapeutic antibodies (e.g., ¹³¹I-ch81C6) to tumorous stroma. This is because the unlabeled ch81C6 binds the normal, healthy stromal sites of tenascin expression, thus protecting these sites from the radio-labeled anti-stromal antibody and increasing delivery to the tumorous sites.

Trial.

This trial builds on previous work with escalated doses of ¹³¹1-ch81C6 antibody delivered without any associated treatment with an unlabeled antibody to potentially “block” binding to normal, healthy stroma and non-specific binding to other healthy tissue. A first trial identified a 10 mCi “dosimetric dose” (of ³¹¹I) followed by a 30 mCi therapeutic dose as the maximum tolerated dose (MTD), due to hematopoietic suppression (this is the suppression of the formation of blood cells, resulting in thrombocytopenia (a decrease in the number of platelets), anemia (a decrease in the number of red blood cells), and neutropenia (a decrease in the number of white blood cells, especially neutrophils) when no “blocking” antibody is used.

Purpose.

The primary objective of this trial is to estimate the maximum tolerated dose (MTD) in terms of toxicities of unlabeled chimeric 81C6 anti-tenascin human/chimeric monoclonal antibody (ch81C6) delivered intravenously in subjects with lymphoma or a solid tumor prior to a fixed radio-labeled dose. A secondary objective is to evaluate the pharmacokinetics and dosimetry of ¹³¹I-labeled ch81 C6 anti-tenascin monoclonal antibody following escalated doses of unlabeled antibody. The secondary goal of the study is to evaluate the potential anti-lymphoma effects of ¹³¹I-labeled ch81C6 anti-tenascin monoclonal antibody.

Test Group.

The test group can include adult subjects that have a biopsy-proven lymphoma or solid tumor and have failed at least one regimen of therapy. Pregnant or lactating women are ineligible. Subjects must not have significant medical and/or psychiatric illness which may compromise any aspect of the planned treatment. Subjects must have adequate (either high enough or low enough) laboratory-determined levels of white blood cells, neutrophils, platelets, transaminases (AST/ALT test), alkaline phosphatase, bilirubin, and creatinine clearance). Subjects with greater than 25% liver and/or bone marrow involvement with disease are not eligible. Subjects must have a performance status of less than 2 and a life expectancy of at least 2 months. Subjects must have the ability to have stem cells harvested. Subjects must be HIV and active viral hepatitis (HBV- and HCV-caused) negative. Subjects previously treated with autologous (from “self”) or allogeneic (from another human) bone marrow transplantation are ineligible. Subjects must be able to receive radiation. Subjects allergic to iodine are excluded. Subjects who are currently undergoing steroid therapy are permitted, though they should be receiving the lowest possible dose and the dose should be stabilized for at least 5 days before therapy is administered.

Test Protocol.

Thyroid suppression will begin at least 24 hours prior to treatment and continue for 14 days after the therapeutic dose. Though the cold (unlabeled) antibody is not known to cause toxicity in humans, 81C6 antibody toxicity will be evaluated during this trial. The dose of therapeutic antibodies (i.e, those that are radio-labeled) will be set at 16% below the MTD determined previously (25 mCi rather than 30 mCi),in the event that there is additional toxicity from the cold antibody.

Subjects are enrolled and treated in this trial in cohorts containing a minimum of there subjects. Each subject will first receive the lowest dose of unlabeled ch81C6 anti-tenascin antibody. If no subjects experience dose-limiting toxicity (“DLT”) at the lowest dose, the dose will be escalated to the next dosage level. If one subject in any three-subject cohort experiences DLT, then three additional subjects will be added to that cohort at the same dose level. If less than two of these six subjects experience DLT, then the dose will be escalated to the next dosage level. However, if two or three subjects in any three-person cohort experience DLT at a certain dosage level, then the MTD is determined to have been exceeded, and the trial will be closed at that time. The MTD will be estimated as the next lower dose of unlabeled antibody used in this trial (i.e., the highest dose given that did not exceed the MTD). If we reach the maximum planned unlabeled dose, which is 1600 mg, without reaching the MTD, the trial may be amended depending on the dosimetry evaluations and approved by the IRB and FDA prior to further escalation Test subjects are first given an unlabeled dose of antibody immediately, immediately followed by a tracer amount (a “tracer amount” is an amount sufficient to localize the antibody through the use of scintigraphy or some analogous method)of 5 mCi of ¹³¹I conjugated to a constant dose of 10 mg of ch81C6 MAb. This is termed the “dosimetric dose.” Because of slight variations in yield and transfer of radio-labeled protein to injection syringes, the dosimetric dose may be adjusted to 10 mg plus 2 mg. The administered, or therapeutic, radiation dose may vary 10% from the targeted dose, for the same reasons.

Subjects will have blood drawn for pharmacokinetic measurements at the following time intervals: 1) immediately after the radio-labeled antibody (dosimetric dose) infusion; 2) at 2 to 5 hours after the dosimetric dose; 3) at approximately 24 hours after the dosimetric dose; 4) once at 2 to 3 days after the dosimetric dose; and 5) once between 4 to 7 days following infusion of the dosimetric dose, until adequate dosimetry analysis is completed. Further testing may be needed if a subject retains the radioactivity longer.

Gamma camera whole body imaging will be performed at all the above-mentioned time points, i.e, whenever blood is drawn. Again, further testing may be needed if a subject retains the radiolabel longer. From these data, the activity of the radiolabel ch8 I C6 will be determined,. From this, the total body exposure dose that will be delivered with the dose of blocking antibody will be determined. This will be followed by the therapeutic radio-labeled dose.

Blood is collected for human-anti-mouse-antibodies (HAMA) testing prior to dosimetry, prior to the therapeutic dosing, and at the time of recovery (restaging). Subjects may have a biopsy of the marrow performed once during the dosimetric week to allow a direct reading of the radioactivity. The results of the biopsy can be correlated with the scanned images to allow a sensitivity calculation for the scans, which in turn allows for an absolute radioactivity scale to be determined (rather than a relative scale, which is all that would be possible without a biopsy). Subjects may have a peripheral node greater than 1 cm that is palpable or be amendable to a bone marrow biopsy following dosimetry. They will be asked to allow an additional biopsy of this site to assist in confirming the dosimetry and pharmacokinetic modeling.

Subjects return for the “therapeutic dose” infusion approximately 1 to 2 weeks after the initial dose. The same unlabeled dose of antibody will be delivered prior to the “therapeutic dose” of 25 mCi attached to 10 mg anti-tenascin on a constant dose of 10 mg of ch81C6 MAb. The therapy comprises a one-time dose event.

Those who experience prolonged cytopenia will have their back-up stem cells thawed and re-infused to assist in recovery in the event that no signs of recovery are noted. Subjects will receive antibiotics, nutritional support, hydration and other supportive care per the treating physician's discretion. Subjects may receive G-CSF (Granulocyte Colony Stimulating Factor) or GM-CSF (Granulocyte-Macrophage Colony Stimulating Factor) at the treating physician's discretion as long as it is not within 7 days following the therapeutic dose of radio-labeled antibody (about one half the life of the therapy).

Data Analysis and Monitoring.

Data is analyzed separately for the 2 different test groups: 1) subjects with lymphoma; and 2) subjects with a solid tumor.

DLT is defined as grade 3 toxicity lasting longer than 1 week, grade 4 for longer than 2 days, or life-threatening non-hematopoietic toxicity using NCI version 3.0 criterion in the pulmonary, renal, hepatic, cardiac, gastrointestinal or neurologic areas, or prolonged cytopenia.

The estimated MTD dose in this trial is the maximum dose of unlabeled antibody delivered prior to the fixed dose of radio-labeled antibody that produces 0 of 3 or<2 of 6 subjects experiencing a dose limiting toxicity.

Production of Immunoglobulin in a Hollow Fiber System.

Hybridoma cells were grown in a Mini-Max™ hollow fiber cartridge system from Biovest International (Worcester, Mass.). The cartridge has 2.3 m² of surface area, a 30,000 MW cutoff, and 80 ml extra capillary space. Cells were cultured in CD Hybridoma Medium from Invitrogen (Carlsbad, Calif.), as follows: 1 to 3×10⁹ ch81C6 hybridoma cells were seeded into the one extra capillary space and their metabolic activity was monitored by measuring the level of lactate, using a Lactate kit from Sigma Chemical Company (Cat # 735-10; St. Louis, MO). The immunoglobulin content of the extra capillary space was monitored on a Biacore 3000 SPR (Surface Plasmon Resonance) unit (Biacore, Piscataway, N.J.). Harvested media was centrifuged at 3000×g for 20 minutes to remove cells and stored at 4 degrees Centigrade (4° C.). Harvested supernatants were pooled and centrifuged at 100,000×g for 30 minutes and then filtered through a Millipak 60 0.22 micron filter (Millipore).

Purified Monoclonal Anti-Tenascin Antibody ch81C6.

Filtered ch81C6 MAb was adjusted to pH 8.0 by adding 10 ml sterile 1.0 M Tris buffer, pH 8.0, per liter of culture media. Chimerized immunoglobulin is purified from culture media by passing over a protein-A Sepharose column that is sterilized by flushing with 10 column volumes of 4 M guanidine-HCl. After rinsing column with 10 column volumes of Tris-NaCl buffer (10 mM Tris in 0.9% NaCl, pH 8.0), culture media is passed through the protein-A column. Column is rinsed with pH 8.0 Tris-NaCl buffer and bound immunoglobulin is eluted with pH 3.0 glycine HCl buffer (0.55 M glycine, 0.85% NaCl and 10 mM HCl.). Five ml fractions are collected and immediately neutralized with 0.5 ml 1 M Tris. Absorbance is read at 280 nm with a spectrophotometer and the fractions containing immunoglobulin are pooled. Purity of pooled immunoglobulin is checked by gel filtration on a 4.6×300 mm HPLC Super TSK-3000 column and then dialyzed overnight against 20 volumes of 50 mM Tris-acetate buffer (pH 5.6) in 50,000 molecular weight cut off dialysis tubing. Forty-micron size ABx (a mixed-mode ion exchanger) and PEI (polyethyleneimine) are obtained from J T Baker Company (Philipsburg, N.J.) in bulk and appropriately sized columns are packed for the amount of immunoglobulin to be bound. One gram of dry ABx will bind 100 to 200 mg of immunoglobulin under ideal conditions. Stainless steel columns are heated to 210° C. for four hours prior to packing, to remove any endotoxins. Packing material and HPLC (“High Performance Liquid Chromatography”) columns are sterilized by flushing with 10 column volumes of 4 M guanidine. Column is then equilibrated by flushing with 20 to 30 column volumes of 50 mM Tris acetate buffer (pH 5.6). Dialyzed immunoglobulin is injected onto the PEI and ABx columns. Columns are rinsed with 10 column volumes of equilibration buffer and prior to elution of immunoglobulin from the ABx column, the PEI column is removed (endotoxins, but not ch81C6, bind to PEI, so removal of the PEI facilitates removal of any endotoxins present). Elution of bound antibody from the ABx column is accomplished by running a 60 minute linear gradient from 0%/-100% 250 mM potassium phosphate buffer (pH 6.8) and 1 milliliter fractions are collected. Absorbance of eluent is monitored at 280 nm. Tubes containing immunoglobulin are pooled and dialyzed exhaustively against 115 mM phosphate buffer, pH 7.4. To remove endotoxins, dialyzed immunoglobulin is passed over a ActiClean Etox column (Sterogene Bioseparations, Inc., Carlsbad, Calif.) that was sterilized with 1 M NaOH and equilibrated with 115 mM phosphate buffer. Protein concentration of ch81C6 is determined and the antibody solution is aliquoted into sterile and pyrogen-free vials by injecting the solution directly into the vials through a 0.22 micron Millipore filter. Protein concentration is determined after filtering to make sure no protein is lost during filtration.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for dosimetry estimation for a region of interest at or around a surgically created resection cavity (SCRC) in a subject in need thereof, which method comprises:
(a) determining a size of the surgically created resection cavity (SCRC);
(b) administering a dosimetric dose of a radio-labeled antibody into the region of interest;
(c) measuring detected radiation from the region of interest at a plurality of times subsequent to administering the dosimetric dose;
(d) determining a residence time based on the size of the surgically created resection cavity (SCRC) and the measured detected radiation from the region of interest; and
(e) calculating an administered radioimmunotherapy (RIT) dose based on the residence time, the size of the surgically created resection cavity (SCRC), and a predetermined radiation absorbed dose (predetermined RAD).

2. The method of claim 1, further comprising detecting the radiation from the region of interest by performing whole-body scintigraphy.

3. The method of claim 2, wherein the whole-body scintigraphy is performed at a first time that is substantially the same time as administering the dosimetric dose, at a second time that is about twenty-four hours subsequent to the administering of the dosimetric dose, and at a third time that is about forty-eight hours subsequent to the administering of the dosimetric dose.

4. The method of claim 1, further comprising determining the size of the surgically created resection cavity by performing magnetic resonance imaging (MRI).

5. The method of claim 1, wherein the predetermined radiation absorbed dose (predetermined RAD) is a predetermined optimal absorbed dose based on experimental data.

6. The method of claim 1, wherein the predetermined radiation absorbed dose (predetermined RAD) is about 44 Gy.

7. The method of claim 1, wherein the administered radioimmunotherapy (RIT) dose is an amount of ¹³¹I-labeled anti-tenascin murine 81C6 (m81C6) monoclonal antibody.

8. The method of claim 1, wherein the administered radioimmunotherapy (RIT) dose is an amount of ¹³¹I-labeled anti-tenascin human/murine 81C6 (ch81C6) monoclonal antibody.

9. The method of claim 1, wherein the region of interest is a 2 cm-wide area of parenchymal tissue surrounding and encircling the SCRC, beginning at the margin of the surgically created resection cavity (SCRC) and extending about 2 cm out from the margin the surgically created resection cavity (SCRC) .

10. The method of claim 1, wherein the administered radioimmunotherapy (RIT) dose is calculated based on the formula:
$${A}_{0} = \frac{{D}_{SCRC}}{S ( {B}_{2 - cm} ← SCRC ) {τ}_{SCRC}}$$
where D_{SCRC} is the predetermined radiation absorbed dose (predetermined RAD), S(B_{2-cm}←SCRC) is an estimated S-value based on the size of the surgically created resection cavity (SCRC) in Gy hr mCi-1, and τ_{SCRC} is the surgically created resection cavity (SCRC) residence time.

11. The method of claim 1, wherein the administered radioimmunotherapy (RIT) dose is administered using a Rickman reservoir implantation.

12. A method for dosimetry estimation as in claim 1, further comprising the steps of:
(a) providing a computer readable medium having computer executable instructions for calculating the required administered activity, the computer executable instructions for performing the steps of:
(b) receiving a number of parameters necessary to calculate the surgically created resection cavity (SCRC) percent-injected dose, the SCRC residence time and the S-value for the surgically created resection cavity (SCRC) for a given session;
(c) plugging the parameters into predefined formulas to calculate the required administer activity; and
(d) outputting the calculated required administered radioimmunotherapy (RIT) dose to a user display.

13. The method for dosimetry estimation as in claim 12, further comprising computer executable instructions for performing the step of storing the parameters and/or the calculated required administered radioimmunotherapy (RIT) dose in a storage device.

14. A method for dosimetry estimation for a region of interest at or around a surgically created resection cavity (SCRC) in a subject in need thereof, which method comprises calculating an administered radioimmunotherapy (RIT) dose from (i) residence time based on (a) size of the surgically created resection cavity (SCRC) and (b) measured radiation detected at a plurality of times subsequent to administering a dosimetric dose of a radio-labeled antibody administered into the region of interest, and (ii) a predetermined radiation absorbed dose (predetermined RAD).

15. (canceled)

16. (canceled)

17. A method for calculating an administered radioimmunotherapy dose for administration at or around a surgically created resection cavity (SCRC), which method comprises using a formula that calculates an administered radioimmunotherapy (RIT) dose based on a given residence time, a given size of the surgically created resection cavity (SCRC), and a predetermined radiation absorbed dose to calculate the administered radioimmunotherapy (RIT) dose.

18. The method of claim 17, wherein the administered radioimmunotherapy (RIT) dose is calculated based on the formula:
$${A}_{0} = \frac{{D}_{SCRC}}{S ( {B}_{2 - cm} ← SCRC ) {τ}_{SCRC}}$$
where D_{SCRC} is the predetermined absorbed dose, S(B_{2-cm}←SCRC) is an estimated S-value based on the size of the surgically created resection cavity (SCRC) in Gy hr mCi-1, and τ_{SCRC} is the surgically created resection cavity (SCRC) residence time.

19. A method of enhancing delivery of therapeutic antibodies that specifically bind to an extracellular stromal constituent of a tumor in a mammalian subject in need thereof, the method comprising the steps of:
(a) administering to the subject an effective dosage of unlabeled blocking antibodies, the unlabeled blocking antibodies specifically binding to substantially all of non-target extracellular stromal constituent of normal tissue while binding to a substantially small percentage of the extracellular stromal constituent of the tumor, thus blocking the binding of the therapeutic antibodies to the non-target extracellular stromal constituent of normal tissue; and then
(b) administering to the subject a treatment effective amount of the therapeutic antibodies, the therapeutic antibodies being specific for the extracellular stromal constituent of the tumor.

20. The method of claim 19 wherein the blocking antibodies are monoclonal antibodies.

21. The method of claim 19, wherein the therapeutic antibodies are monoclonal antibodies.

22. The method of claim 19, wherein the therapeutic antibodies are coupled to a therapeutic agent.

23. The method of claim 19, wherein the therapeutic antibodies are coupled to a therapeutic agent selected from the group consisting of radionuclides, chemotherapeutic agents, and cytotoxic agents.

24. The method of claim 19 wherein the therapeutic antibodies are conjugated to a radionuclide.

25. The method of claim 19, wherein the therapeutic antibodies are conjugated to a radionuclide selected from the group consisting of ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ¹¹³mIn, ¹¹⁵mIn, ¹²³I, ¹²⁵I, ¹³¹I, ¹⁸⁹Ir, ¹⁹Ilr, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²R. ¹⁸⁶R, ¹⁸⁸Re, ¹⁵³Sm, ⁴⁶Sc, ⁴⁷Sc, ⁷²Se, ⁷⁵Se, ¹⁰Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ¹⁰¹mRh, and ²¹²Pb.

26. The method of claim 19, wherein the therapeutic antibodies are conjugated to a chemotherapeutic agent.

27. The method of claim 19, wherein the therapeutic antibodies are conjugated to a chemotherapeutic agent selected from the group consisting of methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine

28. The method of claim 19, wherein the therapeutic antibodies are conjugated to a cytotoxic agent.

29. The method of claim 19, wherein the therapeutic antibodies are conjugated to a cytotoxic agent selected from the group consisting of ricin, aclacinomycin, diphtheria toxin. Monensin, Verrucarin A, Abrin, Vinca alkaloids, Tricothecenes, and Pseudomonas exotoxin A.

30. The method of claim 19, wherein the extracellular stromal constituent is selected from the group consisting fibrinogen, fibronectin, collagen, laminin, proteoglycan, tenascin, entactin, and thrombospondin.

31. The method of claim 19, wherein the administering step (a) is carried out by intravenous injection.

32. The method of claim 19, wherein the administering step (b) is carried out by injection.

33. The method of claim 19, wherein the administering step (b) is carried out at least 2 days after the administering step (a).

34. The method of claim 19, wherein the administering step (b) is carried out at least 4 days after the administering step (a).

35. The method of claim 19, wherein the subject is afflicted with lymphoma.

36. The method of claim 19, wherein the subject is afflicted with a brain tumor.

37. The method of claim 19, which further comprises monitoring the subject for an adverse reaction to the unlabeled blocking antibodies administered in the step (a), wherein if any subjects experience the adverse reaction to the unlabeled blocking antibodies administered in the step (a), the subjects do not receive the therapeutic antibodies at the administering step (b).

38. (canceled)

39. (canceled)
